(19) United States
(12) Patent Application Publication
(10) Pub. No.: US 2021/0113602 A1
(43) Pub. Date: Apr. 22, 2021

(54) USE OF 6-THIO-2'-DEOXYGUANOSINE (6-THIO-dG) TO TREAT MELANOMA

(71) Applicants: The Board of Regents of the University of Texas System, Austin, TX (US); The Wistar Institute of Anatomy and Biology, Philadelphia, PA (US)

(72) Inventors: Jerry SHAY, Dallas, TX (US); Gao ZHANG, Philadelphia, PA (US)

(73) Assignees: The Board of Regents of the University of Texas System, Austin, TX (US); The Wistar Institute of Anatomy and Biology, Philadelphia, PA (US)

(21) Appl. No.: 16/976,456

(22) PCT Filed: Feb. 28, 2019
(86) PCT No.: PCT/US19/20051

§ 371 Date: Aug. 27, 2020

Related U.S. Application Data
(60) Provisional application No. 62/636,775, filed on Feb. 28, 2018.

(51) Int. Cl. A61K 31/7076; A61K 31/675; A61K 31/155; A61P 35/00

(52) U.S. Cl. CPC A61K 31/7076; A61P 35/00; A61K 31/155; A61K 31/675

(57) ABSTRACT

Targeting RAS is one of the greatest challenges in cancer therapy. Oncogenic mutations in NRAS are present in over 25% of melanomas and patients whose tumors harbor NRAS mutations have limited therapeutic options and poor prognosis. Thus far, there are no clinical agents available to effectively target NRAS or any other RAS oncogene. An alternative approach is to identify and target critical tumor vulnerabilities or non-oncogene addictions that are essential for tumor survival. The inventors investigated the consequences of NRAS blockade in NRAS-mutant melanoma and show that decreased expression of the telomerase catalytic subunit, TERT, is a major consequence. TERT silencing or treatment of NRAS-mutant melanoma with the telomerase-dependent telomere uncapping agent 6-thio-2′-deoxy-guanosine (6-thio-dG), led to rapid cell death, along with evidence of both telomeric and non-telomeric DNA damage, increased ROS levels, and upregulation of a mitochondrial anti-oxidant adaptive response. Combining 6-thio-dG with the mitochondrial inhibitor Gamitrinib attenuated this adaptive response and more effectively suppressed NRAS-mutant melanoma. The newly observed robust dependency of NRAS-mutant melanoma on TERT provides evidence for a new combination strategy to combat this class of tumors, which could be expanded to other tumor types.

This application claims benefit of priority to U.S. Provisional Application Ser. No. 62/636,775, filed Feb. 28, 2018, the entire contents of which is hereby incorporated by reference.

### FIELD

The disclosure relates generally to cancer and more specifically to the use of a telomerase-dependent telomere uncapping agent 6-thio-2′-deoxy-guanosine (6-thio-dG) for the treatment of NRAS mutation cancers.

### BACKGROUND INFORMATION

Significant improvement in the treatment of melanoma has been achieved through the use of targeted- and immuno-therapies6l. Despite this progress, a large percentage of patients do not benefit from these therapies and/or experience disease progression. In particular, melanomas with NRAS mutations are highly resistant to most therapies and have poor prognosis.

NRAS is the second most frequently mutated oncogene in melanoma^{17,18}. In addition to mutations in NRAS, mutations in NF1 (>10%), or activation of receptor tyrosine kinases (RTKs), can also activate RAS signaling in melanoma^{10,36,42}. Furthermore, a frequent mechanism of acquired resistance to BRAF/MEK inhibitors is mediated by secondary mutations in NRAS^{41,58}. Consequently, approximately 40% of melanoma patients have tumors that are driven by aberrant NRAS signaling. Targeting RAS has been remarkably challenging; thus far, there are no drugs in the clinic that directly target mutant NRAS. Alternative approaches, including the use of antagonists of RAS effectors, including RAF and PI3K, have had limited success for the treatment of NRAS-driven metastatic melanoma^{45,27}. Therefore, there is an urgent need to identify vulnerabilities in this tumor type that can be exploited therapeutically.

TERT, the catalytic subunit of telomerase, is a promising therapeutic target for cancer, as it is highly expressed in most tumor cells and seldom expressed in most normal non-transformed cells^{1,24}. Mutations in the TERT promoter have been identified in >70% of melanomas, constituting the most frequent genetic alteration in these tumors^{17,22,19}. These mutations create de novo Ets/TCF (E-twenty-six/ternary complex factor) binding sites, enhancing the expression of TERT in these cells^{17,22}. Clinically, BRAF or NRAS mutant melanoma patients whose tumors have TERT promoter mutations have poor overall survival compared to patients with tumors with a non-mutated TERT promoter¹⁴. These data suggest that TERT is a key player in melanoma and a compelling therapeutic target. In addition to its canonical role in maintaining telomere length, TERT has been recognized to regulate extra-telomeric processes.

For example, TERT has been shown to regulate apoptosis, DNA damage responses, chromatin state, and cellular proliferation. These combined data suggest that TERT-based strategies might have valuable therapeutic effects. Developing clinically relevant approaches to inhibit TERT has been daunting; most TERT inhibitors evaluated thus far target the enzymatic activity of telomerase and rely on critical shortening of telomeres to kill tumor cells; consequently, there is a prolonged lag period for efficacy. This prolonged period could constitute a potential disadvantage, as cancer cells, can rapidly adapt to pharmacological challenges and become resistant. Additionally, the long duration of treatment could lead to increased toxicity and/or decreased tolerability. Hence, novel TERT-based therapeutic strategies that can elicit relatively rapid and sustained effects could have significant impact on cancer treatment.

### SUMMARY

The present disclosure relates to a method of treating a cancer cell or a tumor having an NRAS mutation comprising contacting the tumor with 6-thio-2′-deoxy-guanosine (6-thio-dG). In one aspect, the method further comprises contacting the cancer cells or tumor with a mitochondrial inhibitor either prior to, simultaneous with or following treatment with 6-thio-dG, for example, Gamitrinib. In an illustrative example, herein the cancer is metastatic melanoma.

Administration can be by any route, including but not limited to an oral, intratumoral, intravenous or intrathecal route. Other cancers or tumors that can be treated as described herein include but are not limited to breast, prostate, colon, liver, kidney, melanoma, skin cancer, head and neck, brain, lung, bone, hematopoietic cancer, leukemias, and pancreatic cancer.

The disclosure further provides a pharmaceutical composition comprising 6-thio-dG and a mitochondrial inhibitor and use of this composition for treating NRAS mutation associated cancer.

Other objects, features and advantages of the present disclosure will become apparent from the following detailed description. It should be understood, however, that the detailed description and the specific examples, while indicating particular embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1a-i. Depletion of TERT induces rapid death of NRASmut melanoma cells. (FIGS. 1a-b) NRASmut melanoma cells were transduced with lentiviruses encoding NRAS shRNA (+) or non-targeting empty vector control (−). (FIG. 1a) NRAS levels were assessed by immunoblotting (bottom panel) and TERT mRNA levels were assessed by qRT-PCR. (FIG. 1b) Relative telomerase activity (R.T.A.) was determined by TRAP assays following NRAS depletion in NRASmut melanoma cells. Negative control (−): primer; positive control (+): HCT116 cells. (FIGS. 1c-g) TERT was depleted using two different hairpins (sh1, sh2). Apoptosis was by determined by flow cytometry using the Annexin V analog PSVue in cells transduced with TERT shRNA 10 days post infection (dpi). TERT mRNA levels are shown below the corresponding bar as fold change relative to vector control (FIG. 1c). (FIG. 1d) Telomere length was assessed by Southern blotting of terminal restriction fragments (TRF) in NRASmut cells 10 days after transduction. (−): empty vector, pGIPZ; (+): TERT shRNA. Mean TRF length expressed as kilobase pairs is indicated at the bottom. (FIGS. 1e-g) NRASmut melanoma cells were transduced with TERT shRNA. Telomere dysfunction-induced foci (TIF) was determined 7 dpi by indirect immunofluorescence combined with fluorescence in situ hybridization (FISH). Bottom panel, quantification of TIF assays from two independent experiments. Cells were scored as TIF positive when 4 or more γH2AX foci (green) co-localized with telomere foci (red) (FIG. 1e). DNA damage was assessed by γH2AX staining by flow cytometry (FIG. 1f). ROS levels were measured by CellRox-Deep Red staining by FACS (FIG. 1g). (FIGS. 1h-i) WM3000 cells were transduced with wild-type (WT) or catalytically-impaired VYLF1016 (V1016F), VYFL1028 (N1028W) TERT mutant constructs, followed by shRNA-mediated NRAS silencing. Cell death was assessed by FACS measurement of Annexin V and propidium iodide (PI) staining (FIG. 1h). ROS levels were measured by H2DCFDA fluorescence (FIG. 1i). pLKO.1 and pBlast are empty vectors for NRAS shRNA and TERT constructs, respectively. Data represent average of three independent experiments +/−SEM. *p<0.05; **p<0.01; ***p<0.005 determined by unpaired Student's t-test.

FIGS. 2a-h. 6-thio-dG impairs viability of NRASmut melanoma cells. (FIGS. 2a-c) NRASmut melanoma cells (FIG. 2a), BRAF-V600E/NRAS mutant cells derived from melanoma patients resistant to BRAF/MEK inhibitors (FIG. 2b), or non-transformed melanocytes and fibroblasts (FIG. 2c) were treated with 6-thio-dG for 7 days. Cell viability was measured by Alamar Blue assay and calculated relative to DMSO-treated cells. (FIG. 2d) NRASmut melanoma cells and non-transformed human fibroblasts were treated with 1 or 5 μM 6-thio-dG for 7 or 14 days. Cell death was assessed by PSVUe staining. Percent of apoptotic cells is shown. (FIG. 2e) Cells were treated with DMSO or 1 μM 6-thio-dG for 14 days and telomere length was assessed by Southern blotting of TRF. Mean TRF length expressed as kilobase pairs is indicated at the bottom. (FIGS. 2f-g) Cells were treated with 1 μM 6-thio-dG for 7 days. (FIG. 2f) ROS production was measured by H2DCFDA fluorescence and normalized to MSO treated cells. (FIG. 2g) DNA damage was assessed by γH2AX staining. Data represent average from three independent experiments±SEM. *p<0.05; **p<0.01; ***p<0.005 in unpaired Student's t-test. (FIG. 2h) Mice bearing established WM300 or M93-047 NRASmut tumors were treated with vehicle control or 6-thio-dG (2.5 mg/kg i.p; q.d). Tumor volume was measured and average tumor volume (n=4)±SEM was plotted vs. time.

FIGS. 3a-g. Telomere dysfunction enhances oxidative stress leading to upregulation of an antioxidant program. (FIGS. 3a-d) NRASmut melanoma cells were transduced with TERT shRNA (FIG. 3a, FIG. 3c) or treated with 1 μM 6-thio-dG for 7 days (FIG. 3b, FIG. 3d). mRNA levels of the indicated genes were quantified by qRT-PCR (FIG. 3a, FIG. 3b). Mitochondrial superoxide production was measured by MitoSox Red fluorescence (FIG. 3c, FIG. 3d) and expressed as fluorescence relative to vector or DMSO controls. (FIG. 3e) NRASmut melanoma cells were treated with 1 μM of 6-thio-dG for the indicated times and SOD2 protein levels measured by Western blotting. (FIG. 3f) SOD2 was silenced using shRNA. Transduced M93-047 cells were treated with DMSO or 1 μM of 6-thio-dG and cell death was assessed by Annexin V/PI straining. (FIG. 3g) M93-047 cells were transduced with a SOD2 lentiviral construct or empty vector control (pLX304). Transduced cells were treated with increasing doses (1, 2.5 and 5 μM) of 6-thio-dG for 7 days and cell death was assessed. Data represent average from three independent experiments±SEM. *p<0.05; **p<0.01; ***p<0.005 in unpaired Student's t-test.

FIGS. 4a-e. Induction of telomere and mitochondrial dysfunction triggers cell death in NRAS mutant melanoma. (FIG. 4a) NRASmut melanoma cells transduced with TERT shRNA were treated 7 days post infection with 5 μM Gamitrinib (Gam) for 48 h. Cell death was assessed by Annexin V and PI staining. (FIG. 4b) NRASmut melanoma cells and non-transformed fibroblasts were treated with 1 μM 6-thio-dG for 5 days. At day 5, culture medium was replaced and cells treated with 1 μM 6-thio-dG plus 5 μM Gam for two more days. Cells were stained with Annexin V and PI and percent cell death was determined by flow cytometry. (FIGS. 4c-e) Cells were treated as in FIG. 4b and mitochondrial ROS levels were measured using Mitosox Red by FACS. Data represent average of three independent experiments ±SEM. P values were calculated using unpaired Student's t-test; * p<0.05; ***p<0.005.

FIGS. 5a-e. Combination treatment with 6-Thio-dG and Gamitrinib triggers death selectively in NRAS mutant tumor cells. NRAS-WT/BRAF-WT melanoma cells (FIG. 5a), NRAS-WT/BRAF-mut melanoma cells (FIG. 5b), NRAS mutant glioblastoma cells (FIG. 5c), NRASWT/KRAS-mutant colon cancer cells (FIG. 5d), and NRAS-WT/KRAS-mutant lung cancer cells (FIG. 5e) were treated with 1 μM 6-thio-dG for 5 days. At day 5, culture media was replaced and cells treated with 1 μM 6-thio-dG plus 5 μM Gam for two more days. Cells were stained with Annexin V and PI and percent cell death was determined by FACS. Data represent average of three independent experiments +/−SEM; p values were calculated using un-paired Student's t-test; * p<0.05; ** p<0.001.

FIGS. 6a-e Combining 6-thio-dG and Gamitrinib inhibits the growth of 3D melanoma spheroids and NRAS-mutant xenograft tumors. (FIG. 6a) Collagen-embedded melanoma spheroids were treated with DMSO, 6-thio-dG (5 μM) and Gam (5 μM) as in FIGS. 4a-c. On day 7, spheroids were stained with Calcein AM (live cells;

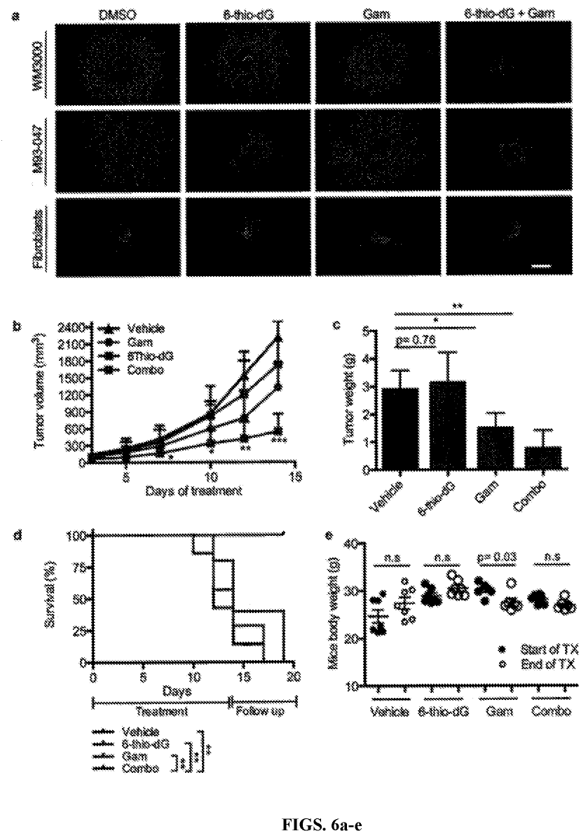

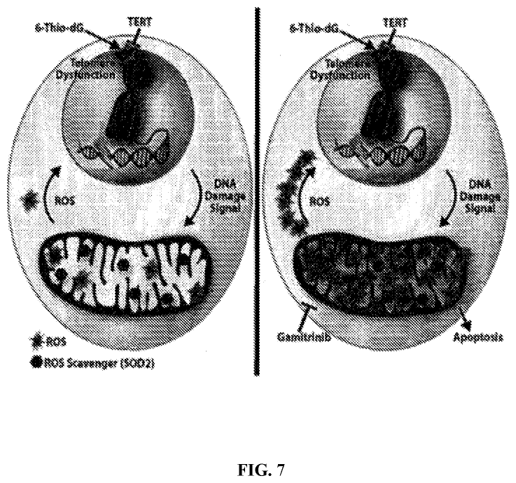

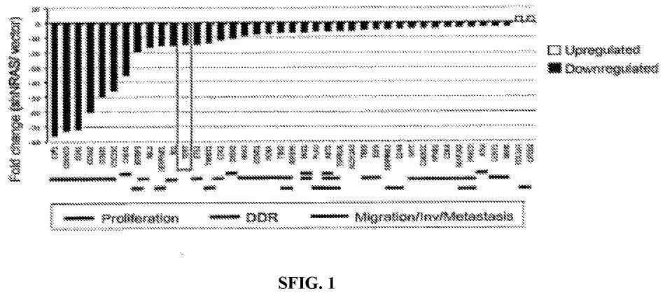

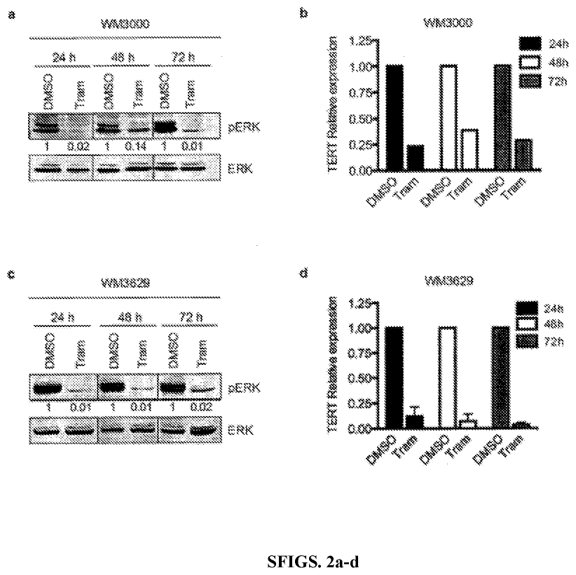

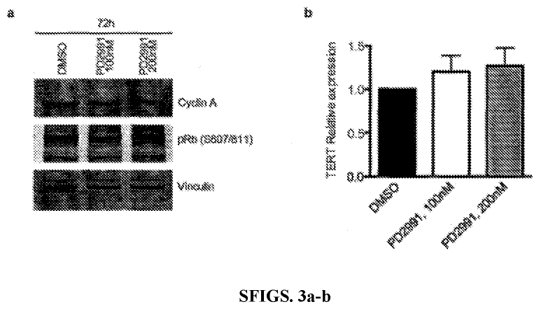

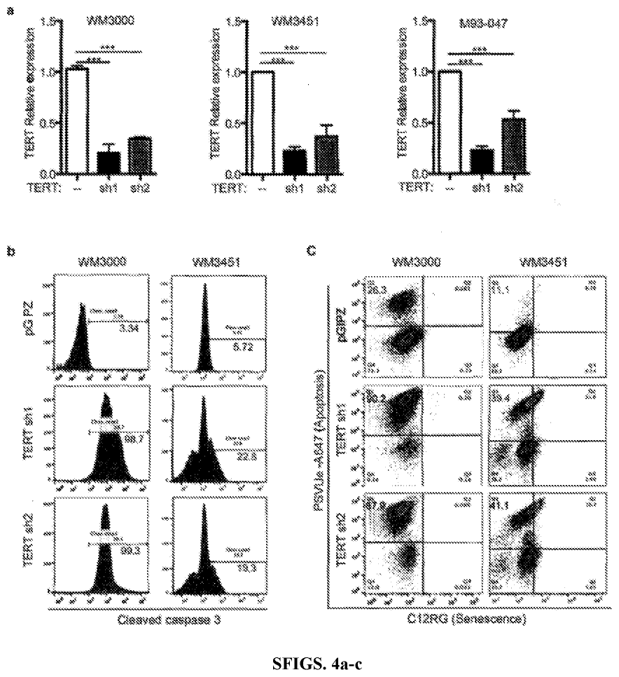

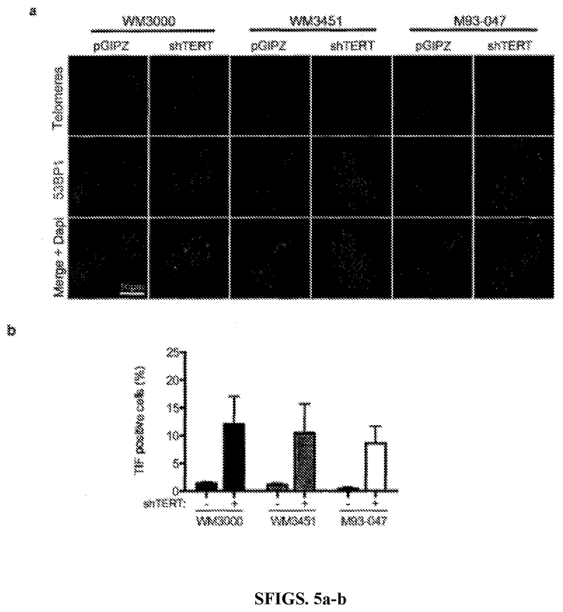

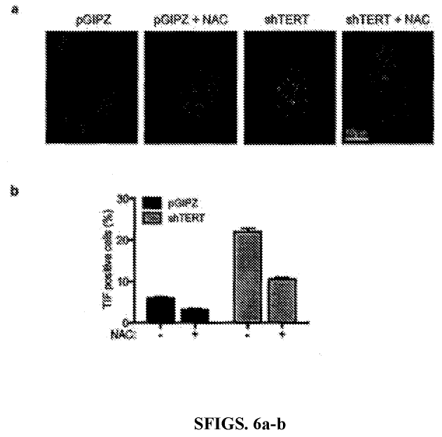

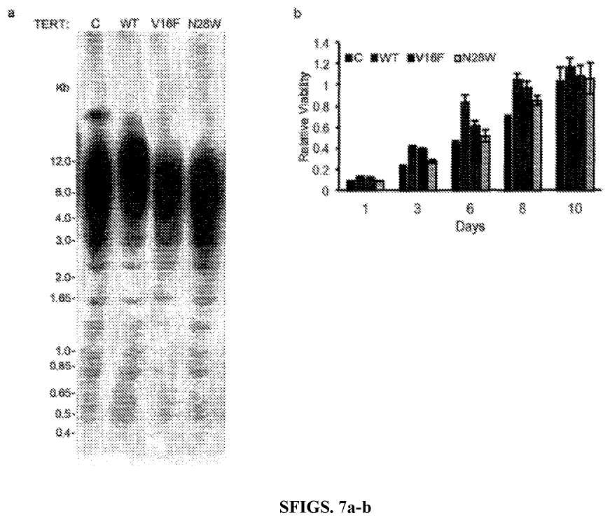

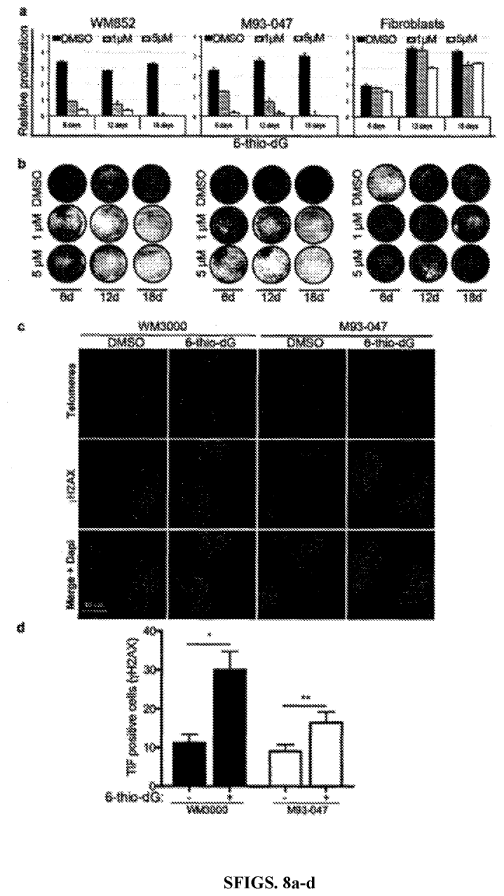

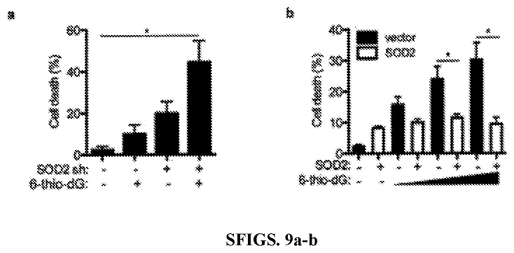

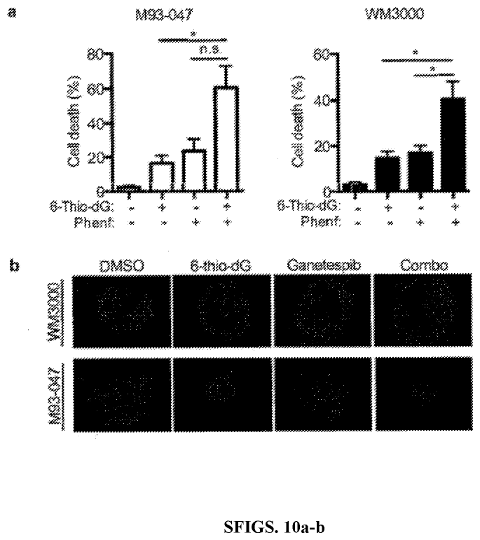

green) and EtBr (dead cells; red) and imaged using an inverted microscope (4×; scale bar=250 μm). Representative merged images from three independent experiments are shown. (FIGS. 6b-d) NRAS mutant M93-047 tumor bearing mice were treated with vehicle control, 6-thio-dG (2.5 mg/kg; ip qd), Gam (12.5 mg/kg; ip qod), or the combination of the two drugs for the indicated days (n=7 mice/group). (FIG. 6b) Average tumor volume over time±SEM is represented. (FIG. 6c) Average tumor weight±SEM after 14 days of treatment. P values were calculated using unpaired Student's t-test. * p<0.05, **p<0.01, ***p<0.005, when comparing vehicle control vs. combination treatment groups. (FIG. 6d) Treatment was discontinued after 14 days, and mice were followed until tumor volume reached a preset volume (1500 mm³). Kaplan Meier survival curves of mice treated for 14 days. p-values were calculated by Mantel-Cox logrank test; * p<0.05, **p<0.01, ***p<0.005. (FIG. 6e) Animal weight (grams) before treatment (start of TX) and at the end of the study (End of TX) was assessed and recorded for every mouse enrolled in the study. Mean and SEM are depicted (n=7 mice/treatment group). Statistical significance was assessed by unpaired Student's t-test.

FIG. 7. TERT offsets oxidative stress in NRAS mutant melanoma. TERT is required for telomere integrity. Genetic silencing of TERT or pharmacological induction of telomere uncapping due to incorporation of the nucleoside analogue 6-thio-dG triggers rapid telomere-induced foci (TIF) coupled to global DNA damage and increased production of ROS. Enhanced ROS levels prompts the activation of a ROS scavenging adaptive response, mediated mainly by SOD2, which reestablishes steady levels of ROS and offsets oxidative stress. Treatment of NRAS mutant melanoma cells with Gamitrinib, a mitochondrial disrupting agent, attenuates SOD2 levels impairing its ability to effectively restore redox balance, resulting in toxic levels of ROS and tumor cell death.

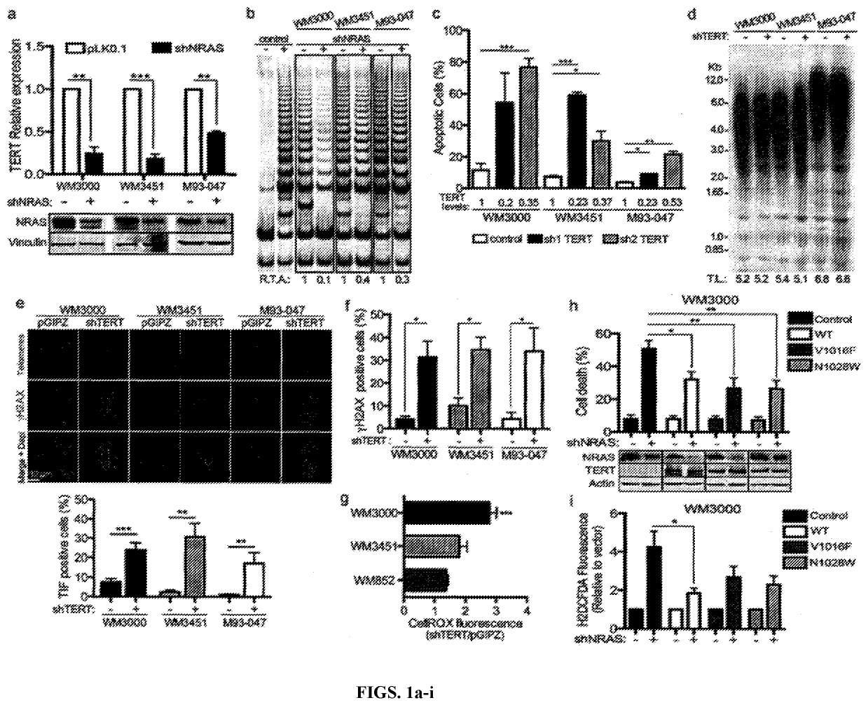

Supplementary FIG. 1. NRAS silencing is coupled to downregulation of TERT. NRAS-mutant WM3000 cells transduced with lentiviruses encoding NRAS shRNA were analyzed using the human cellular senescence PCR array profiler. mRNA levels of the indicated genes were assessed by qRT-PCT following NRAS knockdown.

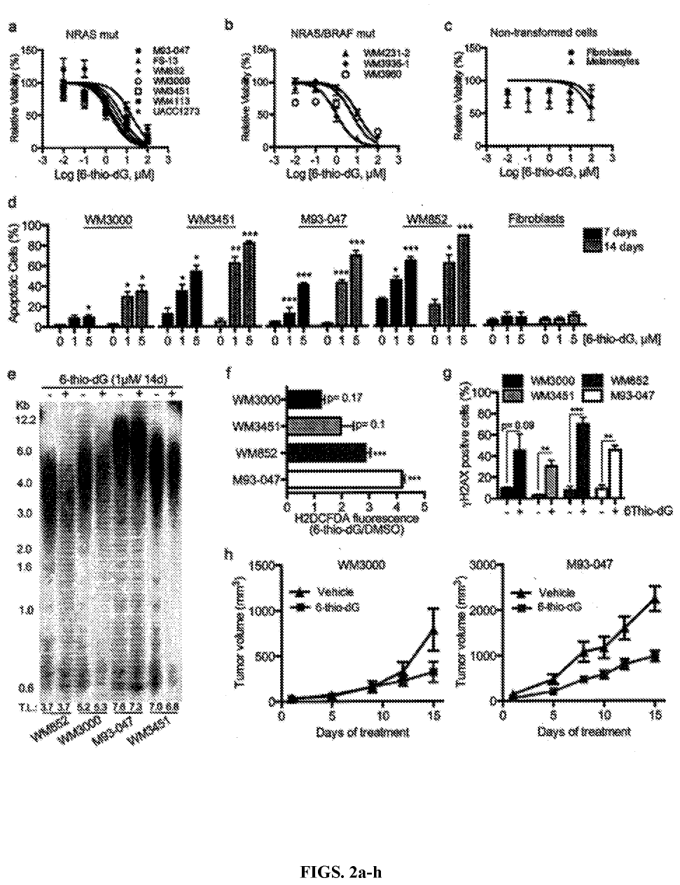

Supplementary FIGS. 2a-d. MEK inhibition downregulates TERT levels. NRAS mutant melanoma cells WM3000 (Supplementary FIGS. 2a, 2b) or WM3629 (Supplementary FIGS. 2c, 2d) were treated with the MEK inhibitor trametinib (100 nM) for 24, 48, 72 h. Inhibition of phosphor-ERK was assessed by immunoblotting. Dotted lines indicated where membranes were cut to remove non-relevant lanes (Supplementary FIGS. 2a, 2c). Relative expression of TERT mRNA levels was determined by qRT-PCR (Supplementary FIGS. 2b, 2d). Data represent average of triplicates +/−SD.

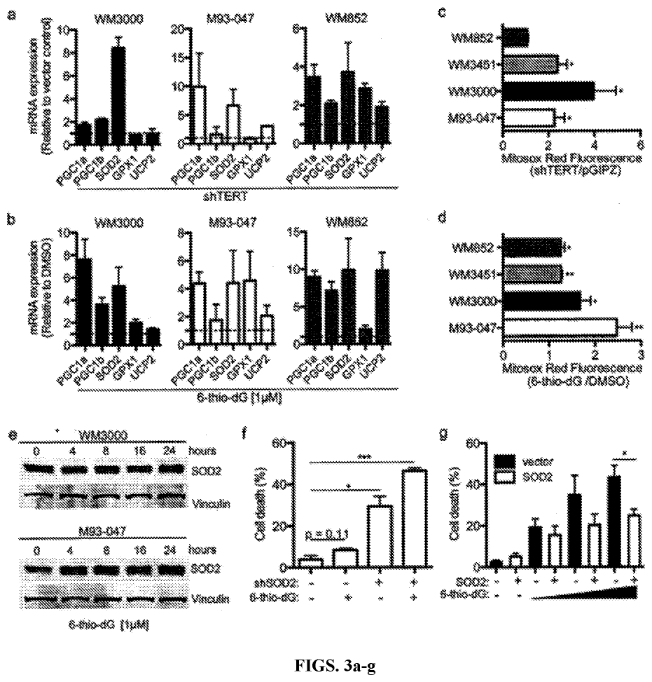

Supplementary FIGS. 3a-b. Inhibition of cdk4/6 does not affect TERT expression. NRAS mutant melanoma cells WM3000 were treated with the cdk4/6 inhibitor palbociclib at the indicated doses for 72 h. (Supplementary FIG. 3a) Downregulation of phosphor-Rb and cyclin A was evaluated by immunoblotting as a surrogate of cell cycle arrest. (Supplementary FIG. 3b) Relative expression of TERT mRNA levels was determined by qRT-PCR. Data represent average of triplicate samples.

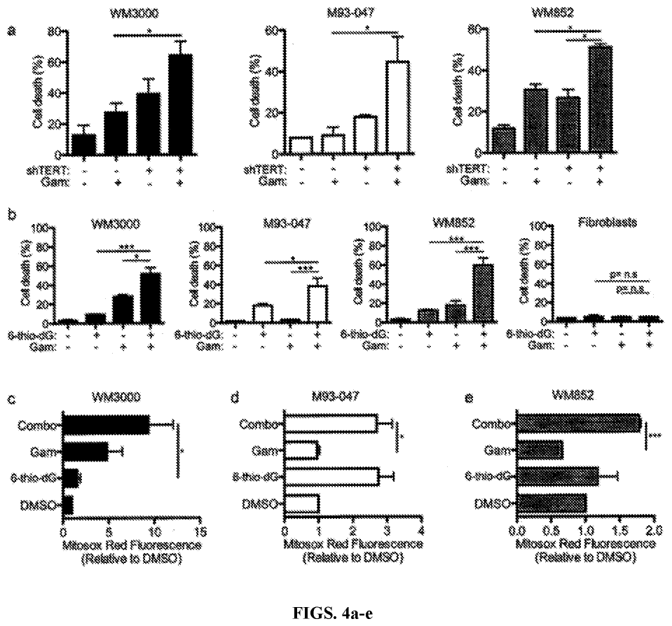

Supplementary FIGS. 4a-c. TERT depletion triggers apoptosis. (Supplementary FIGS. 4a-c) NRASmut melanoma cell lines were transduced with lentiviruses encoding TERT shRNA using two different hairpins (sh1, sh2) or non-targeting empty vector pGIPZ and analyzed 11 days post-infection (dpi). TERT mRNA levels were assessed by qRT-PCT. Data shown present (Supplementary FIG. 4a). Apoptosis was determined using the Annexin V analog PSVue and sensescence was assessed using the ImaGene Red β-galactosidase substrate C12RG by FACS (Supplementary FIG. 4b). Apoptosis was also assessed by cleaved caspase 3 staining (Supplementary FIG. 4c).

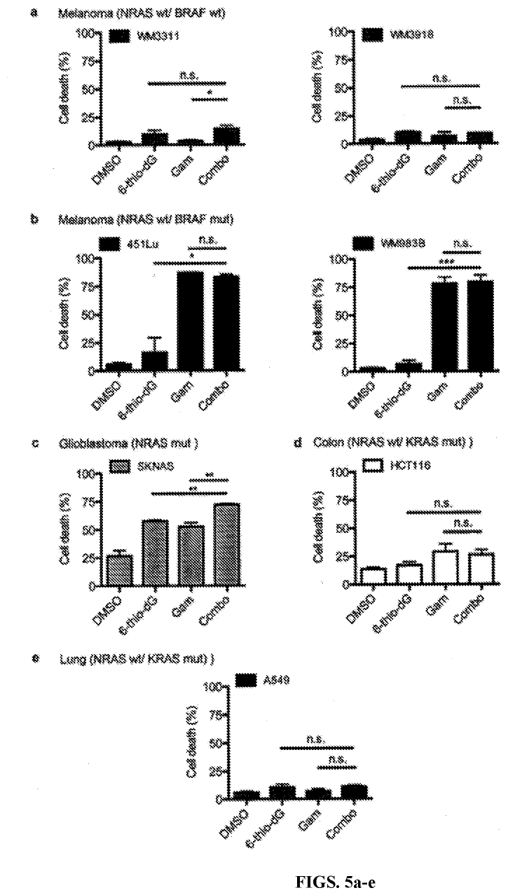

Supplementary FIGS. 5a-b. TERT silencing triggers rapid telomere dysfunction-induced foci. (Supplementary FIG. 53a) Telomere-induced foci (TIF) were determined by indirect immunofluorescence combined with fluorescence in situ hybridization (FISH) in NRASmut cells transduced with TERT shRNA 7dpi. Cells were considered TIF-positive when four or more co-localizing foci of telomeres (red) and 53BPI (green) were found. (Supplementary FIG. 5b) Quantification of TIF-positive cells from two independent experiments. Data are average of two independent experiments +/−SEM.

Supplementary FIGS. 6a-b. Treatment with the antioxidant N-acetyl-L-cysteine (NAC) attenuates TIFs. NRAS mutant melanoma cells WM3000 were transduced with TERT shRNA and treated with NAC (mM) for 7 days. (Supplementary FIG. 6a) TIFs were assessed by indirect immunofluorescence combined with fluorescence in situ hybridization (FISH). Representative images are shown (scale bar=10 μm). (Supplementary FIG. 6b) Quantification of TIF positive cells. Cells were scored as TIF positive when three or more y-H2AS foci (green) were co-localizing with telomere foci (red). Data represent average of thirteen fields imaged +/−SD.

Supplementary FIGS. 7a-b. Catalytically-impaired TERT mutants lead to telomere attrition in NRAS mutant melanoma cells. (Supplementary FIG. 7a) Telomere length was assessed by Southern blotting of terminal restriction fragments (TRF) in WM3000 cells transduced with vector control, wild-type (WT), or catalytic-impaired hTERT mutant constructs VYLF1016 (V16F) or VYFL1028 (N28W). (Supplementary FIG. 7b) Relative cell viability was determined by MTT assays at days 1, 3, 6, 8 and 10 post-transduction. Cell viability was calculated relative to vector control-transduced cells.

Supplementary FIGS. 8a-d. NRAS mutant melanoma cells are sensitive to the telomere uncapping agent 6-thio-dG. NRAS^{mut} melanoma cells were treated with 6-thio-dG for 6, 12 or 18 days. (Supplementary FIG. 8a) Relative cell number was assessed by Cyrstal Violet assay and calculated relative to DMSO-treated cells. Data represent average of three independent experiments +/−SD. (Supplementary FIG. 8b) Representative pictures of cells treated with 6-thio-dG for the indicated days and stained with Crystal Violet. (Supplementary FIG. 8c) NRAS^{mut} cells were treated with 6-thio-dG for 7 days and telomere-induced foci (TIF) were determined by by indirect immunofluorescence combined with fluorescence in situ hybridization (FISH). Cells were considered TIF-positive when for or more co-localizing foci of telomeres and γ-H2AX were found. (Supplementary FIG. 8d) Quantification of TIF assay. Cells were scored as TIF-positive when four or more γ-H2AX foci (green) were co-localizing with telomere foci (red). Data represent average of +/−SEM. P values were calculated by unpaired Student's t-test.

Supplementary FIGS. 9a-b. SOD2 modulates the response to 6-thio-dG.

(Supplementary FIG. 9a) SOD2 was silenced using shRNA. Transduced WM3000 cells were treated with DMSO or 1 μM of 6-thio-dG and cell death was assessed by Annexin V/PI staining. (Supplementary FIG. 9b) Cells were transduced with a SOD2 lentiviral construct or empty vector control (pLX304). Transduced cells were treated with increasing doses (1, 2.5 and 5 μM) of 6-thio-dG for 10 days and cell death was assessed. Data represent average from two independent experiments +/−SEM. *p<0.05 in unpaired Student's t-test.

Supplementary FIGS. 10a-b. Impairing mitochondrial function potentiates the effects of 6-thio-dG. (Supplementary FIG. 10a) NRASmut melanoma cells were treated with 6-thio-dG (1 μ) for 7 days total. At day 5, culture medium was replaced and cells were treated with 1 μM 6-thio-dG plus 1 mM Phenformin for two more days. Percent cell death was determined by flow cytometry following staining of cells with Annexin V and Propidium iodide. Data represent average of three independent experiments +/−SEM. *p<0.05 in unpaired Student's t-test. (Supplementary FIG. 10b) Collagen-embedded spheroids were treated with DMSO, 6-thio-dG (5 μM) and Ganetespib (5 μM) as in

Supplementary FIG. 10a. On day 7, spheroids were stained with Calein-AM (live cells; green) and EtBr (dead cells; red) and imaged using an inverted microscope (4X). Representative merged images from three independent experiments are shown.

### DETAILED DESCRIPTION

Here, the inventors hypothesized that resistance to TERT inhibition depends on activation of an adaptive response, which can be exploited for drug combination strategies providing novel avenues to combat NRAS-driven melanoma. The inventors found that combining 6-thio-dG with the mitochondrial inhibitor Gamitrinib attenuated this adaptive response and more effectively suppressed NRAS-mutant melanoma. This study uncovers a robust dependency of NRAS-mutant melanoma on TERT and provides proof of principle for a new combination strategy to combat this class of tumors, which could be expanded to other tumor types.

### Results

To identify specific vulnerabilities of NRAS mutant melanoma, the inventors performed gene expression analysis in NRAS mutant melanoma cells following depletion of NRAS. The inventors focused on genes known to regulate proliferation and senescence, as they had established that NRAS silencing rapidly triggered proliferation arrest and induced senescence. One of the most pronounced effects of NRAS silencing was downregulation of the catalytic subunit of telomerase, TERT (FIG. 1a; supplementary figures). Of note, TERT levels were downregulated following NRAS depletion in both NRAS mutant melanoma cells harboring TERT promoter mutations and to a lesser degree in melanoma cells with wild-type TERT promoter (Supplementary Table 1). Downregulation of TERT was coupled to diminished telomerase activity (60-90%; FIG. 1b). Consistent with previous reports indicating that the RAS/MEK signaling pathway regulates TERT expression treatment of NRAS mutant melanoma cells with the MEK inhibitor trametinib downregulated TERT mRNA levels (supplementary figures). Since telomerase activity is associated with cell cycle progression, the inventors wondered if the decrease in TERT levels could be the result of a bystander effect of the cell cycle arrest elicited by NRAS silencing. To rule out this possibility, they treated NRAS mutant melanoma cells with a cdk4/6 inhibitor to induce cell cycle arrest and assessed TERT levels. Treatment with the cdk4/6 inhibitor palbociclib (PD-0332991) led to sustained proliferation arrest but did not significantly affect TERT levels (supplementary figures).

To determine the dependency of NRAS mutant melanomas on TERT, the inventors evaluated the effect of TERT silencing in these cells (FIG. 1c). Depletion of TERT, using two different short hairpins, led to extensive and relatively rapid (7-10 days) induction of apoptosis in NRAS mutant melanoma cells, with no evidence of senescence within this time frame (FIG. 1c and supplementary figures). Additionally, the inventors did not detect a significant decrease in average telomere length within 10 days following transduction of shTERT (FIG. 1d), when much of cell death (˜70%) occurred. TERT depletion led to increased telomere dysfunction-induced foci (TIF), increased phosphorylation of histone γ-H2AX and accumulation of reactive oxygen species (FIGS. 1e-g and supplementary figures). To determine if oxidative stress was contributing to telomere dysfunction, cells transduced with non-targeting vector or TERT shRNA were treated with the antioxidant N-acetyl-L-cysteine (NAC). Treatment with NAC attenuated the number of TIF in TERT depleted cells, suggesting that oxidative stress contributes to telomeric DNA damage and dysfunction (supplementary figures). Together, these data support the premise that TERT depletion may lead to both telomeric and non-telomeric DNA damage in NRAS mutant melanoma cells. The inventors next sought to clarify the requirement for the catalytic activity of TERT for the pro-survival function of this protein in NRAS-mutant melanoma. Toward this end, they compared the ability of ectopically expressed wild-type TERT (WT; catalytically active) with two different catalytic domain deficient constructs of TERT for their ability to rescue cell death induced by NRAS silencing. These two mutants, FVYL1016 and FVYL1028, contain point mutations in the FVYL motif, which impair its catalytic activity and lead to telomere attrition in primary human fibroblasts4 and melanoma cells ectopically expressing these constructs (supplementary figures). Of note, mutations within this motif do not affect proper protein folding or proliferation of NRAS mutant melanoma cells ectopically expressing these constructs (supplementary figures). Ectopic expression of wild-type (WT) TERT, or either of the two catalytically impaired TERT mutants, FVYL1016 or FVYL1028, partially protected melanoma cells from cell death induced by NRAS depletion (FIG. 1h). Similarly, ectopic expression of either WTTERT, or to a lesser extent either of the two TERT mutants could alleviate the increased levels of ROS associated with NRAS silencing (FIG. 1i). These combined data indicate that TERT contributes to survival of NRAS mutant melanoma and raises the possibility that, in addition to its catalytic role, TERT might also possess a non-catalytic survival role in melanoma.

To determine the therapeutic value of exploiting melanoma cell dependency on TERT, the inventors used the nucleoside analog 6-thio-dG-2′-deoxyguanosie (6-thio-dG) 39. 6-thio-dG is a telomerase substrate precursor that is rapidly incorporated into the telomeres of cells expressing telomerase, acting as an uncapping agent and leading to rapid induction of TIFs 6-thio-dG has no significant effects on red blood cells, white blood cells, alanine aminotransferase (ALT), aspartate aminotransferase (AST), and creatinine levels in mice. Treatment with 6-thio-dG impaired the viability of NRAS mutant melanoma cells (FIGS. 2a-b and supplementary figures), including cells driven by secondary mutations in NRAS derived from melanoma patients with acquired resistance to BRAF and MEK inhibitors (FIG. 2b). As expected, 6-thio-dG did not significantly affect telomerase negative cells such as non-transformed primary human melanocytes or fibroblasts (FIG. 2c). Treatment of NRAS mutant melanoma cells with 6-thio-dG led to cell death after 7-14 days (FIG. 2d), without evidence for significant telomere shortening after 14 days (FIG. 2e). Similar to the effects of TERT depletion, treatment with 6-thio-dG led to increased levels of ROS (FIG. 20, TIFs (supplementary figures), and γH2AX (FIG. 2g), thus phenocopying the effects of TERT silencing. Further, treatment of NRAS mutant tumor bearing mice with 6-thio-dG slowed the growth of tumors (FIG. 2h).

The inventors noted that TERT depletion or treatment with 6-thio-dG led to upregulation of several detoxifying enzymes such as SOD2, GPX1 and UCP2 (FIGS. 3a-b), as well as other enzymes involved in mitochondrial function and redox balance. Consistently, TERT depletion or treatment with 6-thio-dG led to increased mitochondrial ROS levels (FIGS. 3c-d). The inventors next sought to determine whether the most consistently upregulated anti-oxidant enzyme, SOD2 (FIGS. 3a, 3b and 3e), could be counteracting oxidative stress associated with telomere uncapping, and enabling tumor cells to survive with high levels of ROS. Depletion of SOD2 enhanced the ability of 6-thio-dG to induce apoptosis of NRAS mutant melanoma cells (FIGS. 3f and supplementary figures). Conversely, overexpression of SOD2 attenuated the apoptosis-inducing effects of 6-thio-dG (FIGS. 3g and supplementary figures). Based on the results described above, the inventors hypothesized that combining a mitochondrial disrupting agent with 6-thio-dG or TERT depletion could kill NRAS mutant melanoma cells by concurrently inducing high levels of ROS and blocking the antioxidant adaptive program induced by telomere dysfunction. The inventors tested this hypothesis using Gamitrinib, an ATPase antagonist that disrupts mitochondrial function by selectively inhibiting mitochondrial Hsp90²⁹. Importantly, Gamitrinib has been shown to downregulate SOD2 levels 54; accordingly, depletion of TERT enhanced the cytotoxic effects of Gamitrinib (FIG. 4a). Additionally, pre-treatment of NRAS mutant melanoma cells with 6-thio-dG sensitized cells to the cytotoxic effects of Gamitrinib, enhancing cell death (FIG. 4b). This combination had minimal effects on non-transformed cells, such as human primary fibroblasts (FIG. 4b). Additionally, 6-thio-dG in combination with Gamitrinib led to increased levels of mitochondrial ROS in NRAS mutant melanoma cells (FIGS. 4c-e).

To determine if Gamitrinib could potentiate the efficacy of 6-thio-dG in melanoma cells lacking NRAS mutations, the inventors treated NRAS wild-type melanoma cells with the nucleoside analog and the mitochondrial inhibitor as single agents or in combination (FIGS. 5a-d). They found that melanoma cells lacking NRAS or BRAF mutations (WT/WT) were resistant to 6-thio-dG and Gamitrinib as single agents or in combination, as these compounds did not induce cell death (FIG. 5a). As the inventors have previously shown, BRAFmutant melanoma cells were highly sensitive to Gamitrinib 6; while Gamitrinib alone led to 75-80% cell death, the combination with 6-thio-dG did not further enhanced death of BRAF mutant melanoma cells (FIG. 5b). As the aforementioned data indicated that Gamitrinib selectively potentiates the efficacy of 6-thio-dG in NRAS mutant melanoma cells, the inventors wondered if this combination could trigger cell death in other RAS mutant tumor cells. To answer this question, they tested the effects of Gamitrinib and 6-thio-dG in KRAS mutant lung and colon cancer cell lines as well as NRAS mutant glioblastoma cells. Interestingly, 6-thio-dG and Gamitrinib triggered significant death of NRAS mutant SKNAS glioblastoma cells, as single agents or in combination (FIG. 5c). In contrast, 6-thio-dG and Gamitrinib did not induce substantial death of KRAS mutant HCT116 and A549 cells (FIGS. 5d-e). These data suggest that Gamitrinib potentiates the efficacy of 6-thio-dG selectively in NRAS mutant tumor cells.

To further evaluate the efficacy of 6-thio-dG in combination with Gamitrinib, the inventors treated NRAS mutant melanoma cells grown as 3D-collagen embedded spheroids, which more closely mimic the in vivo behavior of melanoma. Pretreatment of NRAS mutant melanoma spheroids with 6-thio-dG markedly potentiated the effect of Gamitrinib, and induced apoptosis of 3D melanoma spheroids (FIG. 6a). Of note, whereas treatment with another mitochondrial inhibitor phenformin resulted in similar cooperativity with 6-thio-dG (supplementary figures), treatment with ganetespib, an inhibitor of cytosolic HSP90, did not substantially enhance the efficacy of 6-thio-dG (supplementary figures). These data support the premise that drug-induced mitochondrial impairment potentiates the effects of 6-thio-dG. Furthermore, mice treated with the combination of 6-thio-dG plus Gamitrinib exhibited significantly smaller tumors, compared to mice treated with either single agent (FIGS. 6b-c). The combination of 6-thio-dG plus Gamitrinib substantially prolonged the survival of tumor bearing mice (FIG. 6d), even after treatment withdrawal. Of note, this combination was well tolerated in vivo and did not significantly affect animal weight (FIG. 6e). Together, these results indicate that therapeutic approaches exploiting melanoma cell addiction to TERT and combating the ability of melanoma to offset oxidative stress would render cancer cells vulnerable to drug-induced cell death.

### Discussion

The discovery of TERT promoter mutations in most melanomas has provided compelling evidence that this gene plays a critical role in this disease, and therefore constitutes a promising therapeutic target. Here, the inventors used NRAS mutant melanoma as a treatment refractory model to investigate the therapeutic value of exploiting melanoma's addiction to TERT and activation of adaptive mechanisms limiting the effects of TERT-based approaches. They found that telomere-induced dysfunction is associated with oxidative damage followed by relatively rapid melanoma cell death in vitro and decreased tumor growth in vivo. However, tumor cells counteract these effects by activating an anti-oxidant program aimed at restoring redox balance. The inventors show that impairing mitochondrial function and blunting the ROS scavenging machinery renders NRAS mutant tumor cells susceptible to excessive ROS levels, leading to tumor cell death.

Despite the critical role of telomerase in cancer, developing effective antitelomerase therapies has been challenging^{24,52}. Only one compound, imetelstat (GRN163L), an oligonucleotide that binds to the RNA subunit of Telomerase TERC, has shown efficacy in myeloproliferative disorders, but has displayed limited activity in solid tumors. Preclinical approaches to inhibit telomerase have been evaluated in melanoma; nevertheless, telomerase inhibitors have shown modest activity as monotherapy and they have not yet been successfully translated to the clinical setting . Interestingly, inactivation of p21 combined with treatment with imetelstat and CP-31398 (to restore p53 activity), repressed melanoma growth ¹⁵, suggesting that telomerase-based combination approaches might lead to enhanced anti-tumor effects. The lack of success developing effective anti-telomerase approaches and the reason why solid tumors respond poorly to telomerase monotherapy remains elusive. This could be in part related to early efforts focusing mainly on targeting the reverse transcriptase activity of telomerase and being heavily reliant on telomere shortening. A shortcoming of this approach is the relatively long lag period needed for efficacy, which could trigger activation of adaptive mechanisms and drug resistance. In these studies, the inventors found that either TERT silencing or treatment with 6-thio-dG led to telomere dysfunction and relatively rapid cell death. Even though the inventors did not detect significant changes in average telomere length within the time frame that cells underwent apoptosis, it is possible that critically short telomeres, commonly present in melanoma cells, become uncapped and dysfunctional, thereby also contributing to cell death. Of note, it has been reported that melanoma cells harbor a subset of critically short telomeres and that the upregulation of telomerase activity associated with TERT promoter mutations does not preclude telomere attrition. In fact, Chiba and colleagues recently demonstrated that reactivation of telomerase via TERT promoter mutations allows for melanomagenesis first by protecting the shortest telomeres rather than by elongating the telomeres, and subsequently by sustaining tumor cell proliferation. These studies and results suggest that anti-melanoma strategies dependent solely on telomere shortening may not have a significant impact on tumor maintenance⁵⁹. In contrast, induction of telomere uncapping and dysfunction appears to have a more rapid effect triggering apoptosis in a telomere length independent manner^{12,39}. In support of this premise, Blasco and colleagues recently showed that induction of acute telomere uncapping by depletion of the shelterin protein TRF1, can restrain tumor growth independently of telomere length in a p53-null K-Ras (G12V)-induced lung carcinoma mouse model. Likewise, it has been shown that ectopic expression of telomerase can protect cells against double-strand DNA damage in a telomere lengthening independent manner. The inventors found that non-toxic doses of the telomere uncapping agent 6-thio-dG as a single agent slowed, but did not completely abrogated tumor growth in vivo, raising the possibility that induction of telomere dysfunction could prompt the activation of adaptive or compensatory survival mechanisms mitigating the effects of the drug.

While resistance to TERT-based approaches has been attributed primarily to engagement of alternative lengthening of telomeres (ALT), little is known about adaptive survival mechanisms triggered by TERT inhibition or telomere dysfunction. The inventors found that whereas telomere dysfunction is associated with oxidative damage, melanoma cells rapidly activate an anti-oxidant response coupled to increased expression of PGC-1α and ROS scavenging enzymes, primarily Mn-superoxide dismutase SOD2 with no evidence of ALT activation. These results led us to postulate that a rational strategy could involve combining 6-thio-dG with agents that further induce oxidative stress and disable the anti-oxidant machinery of tumor cells. The inventors selected Gamitrinib, a mitochondriotoxic small molecule that selectively blocks mitochondrial HSP90 and exhibits broad anti-cancer activity including efficacy in BRAF-mutant melanoma, but limited activity in NRAS mutant cells as a single agent⁶. Notably, melanoma drug resistance can be mediated by mitochondrial adaptive responses^{16,48,62}. Likewise, Hu et al, demonstrated in an ATM deficient lymphoma mouse model that genetic ablation of telomerase caused cell death, but also induced ALT and PGC-1β 21. They further showed that genetic depletion of PGC-1β impaired mitochondria function, enhancing anti-telomerase therapy. Altogether these studies suggest that mitochondrial function might play a major role modulating multi-drug response and cancer cell viability.

The exact mechanism triggering the mitochondrial detoxifying response prompted by TERT depletion or treatment with 6-thio-dG needs to be further investigated. Several lines of evidence suggest that TERT plays other roles independent of its canonical telomere lengthening function³³. For example, TERT has been shown to traffic to mitochondria^{32,49}; however, the relevance of mitochondrial TERT is not fully understood. Previous reports suggest that ROS can modulate TERT intracellular localization and that TERT can bind to mitochondrial DNA, promoting resistance to oxidative stress and increasing cell survival. Consistent with these studies, TERT overexpression attenuates ROS basal levels and diminishes stress-induced ROS generation. The inventors found that both TERT depletion and treatment with 6-thio-dG upregulated ROS and that TERT can potentiate the antioxidant capacity of melanoma cells in a RT and telomere-lengthening independent manner, enabling melanoma cells to survive under conditions of excessive oxidative stress. Since ROS signaling can induce the expression of FOXO transcription factors, FOXO along with the transcriptional co-activator PGC-1α could enhance the expression of detoxifying enzymes genes such as SOD2 in NRAS mutant melanoma cells. These studies support the notion that NRAS mutant melanoma is a prime candidate for TERT-based therapeutic approaches. These data suggest that in addition to a telomere-dependent role of TERT in melanoma, TERT may also possess a telomere lenghtening-independent role promoting melanoma survival, as catalytically impaired and telomere elongating deficient TERT can protect cells from loss of oncogenic NRAS. These results could have therapeutic implications, as they raise the possibility that approaches that solely target the reverse transcriptase activity of TERT could have limited efficacy impairing tumor growth and maintenance.

Altogether, these studies stress the need to develop drug combinations co-targeting not only telomerase's catalytic activity but also other telomere-lengthening independent functions and adaptive resistance mechanisms. These data provide proof-of-principle for this strategy and for developing similar combinations to increase antitumor responses. Finally, it would be important in future studies to further determine whether this approach could also be applied to other NRAS-driven tumors.

### Materials And Methods

Cell culture, viability and cell death assays. All cells were cultured in RPMI-1640 medium (Corning Cellgro, Manassas, Va.) supplemented with 5% fetal bovine serum (FBS) and grown at 37° C. in 5% CO₂. Human fibroblasts (FF2511) were isolated from foreskin samples and grown in RPMI-1640 supplemented with 10% FBS. Cells were seeded in 96-well plates and treated with drugs. 6-thio-dG was purchased from R.I. Chemical Inc (Orange, Calif.). The complete chemical synthesis, HPLC profile, and mass spectrometry of mitochondrial-targeted small molecule HSP90 antagonist, Gamitrinib (GA mitochondrial matrix inhibitors) has been reported57. The Gamitrinib variant containing triphenylphosphonium as a mitochondrial-targeting moiety was used in this study. Cell viability was assessed following 6 hr incubation with 500 μM Alamar Blue (ThermoFisher Scientific, Waltham, Mass.) using an EnVision Xcite Multilabel plate reader (Perkin Elmer, Waltham, Mass.). Cell death was determined by flow cytometry using PSVue-643 (p-1006; Molecular Targeting Technologies, West Chester Pa.) or Annexin V (640919; Biolegend, San Diego, Calif.) and Propidium Iodide (Sigma-Aldrich, St. Louis, Mo.) staining. Samples were analyzed using a BD LSRII flow cytometer (BD Biosciences, San Jose, Calif.) and analyzed using FlowJo Software v10.0.7 (FlowJo, LLC, Ashland, Oreg., USA). Analysis of samples by flow cytometry was performed blindly.

TERT constructs, small hairpin RNA, and lentivirus infection. Lentiviral NRAS shRNA in pLKO1 backbone and TERT shRNA in pGIPZ backbone were obtained from Thermo Scientific. TERT constructs (wild-type, FVYL1016 or FVYL1028) in a pBlast lentiviral vector have been previously described⁵². Lentiviruses were produced by transfection of 293T cells with packaging plasmids (pPAX2 and pMD2.G) along with 4 μg lentiviral shRNA vector using Lipofectamine 2000 reagent (Invitrogen, Waltham, Mass.) following the manufacturer's instructions. Melanoma cells were transduced with virus in the presence of 6 μg/mL polybrene (Sigma-Aldrich) for 18 hr. Transduced cell populations were selected with appropriate antibiotics. shRNA knockdown efficiency was determined by western blot analysis and/or qRT-PCR.

PCR Array. Human cellular senescence RT2 Profiler PCR array (Qiagen, Valencia, Calif.) was used following manufacturer's specifications. Data was analyzed with the SABiosciences PCR Array Data Analysis Template Excel.

Immunobloting. For western blot analysis, total cell lysates were prepared as previously described⁶⁰. Nitrocellulose membranes were incubated overnight with primary antibodies at 4° C., followed by 1 hr incubation with Alexa Fluor-labeled secondary antibodies (IRDye 680LT goat-anti mouse or IRDye 800CW goat-anti rabbit antibodies (LI-COR Biosciences) at room temperature. Fluorescent images were acquired and quantified by LI-COR Odyssey Imaging System.

Telomerase activity assay. Telomerase activity was measured by TRAP assay as previously described⁴⁰. HCT116 cells and lysis buffer were used as positive and negative controls respectively. Telomerase extension products were amplified by PCR and run on 10% non-denaturing acrylamide gel. Typhoon Phosphoimager scanner (Molecular Dynamics, GE Healthcare, Little Chalfont, UK) was used for visualization of gel products.

Telomere length assay. Genomic DNA was prepared using Wizard genomic DNA purification kit (Promega) following manufacturer's instruction. For telomere length and Southern blot analysis, genomic DNA (˜5 μg) was digested with Alu I+Mbo I restriction endonucleases, fractionated in a 0.7% agarose gel, denatured, and transferred onto a GeneScreen Plus hybridization membrane (PerkinElmer). The membrane was cross-linked, hybridized overnight at 42° C. with 5′-end-labeled 32P-(TTAGGG)4 probe in Church buffer (0.5 N Na₂HPO₄ pH 7.2, 7% SDS, 1% BSA, 1 mM EDTA), and washed twice for 5 min each with 0.2 N wash buffer (0.2 N Na₂HPO₄ pH 7.2, 1 mM EDTA, and 2% SDS) at room temperature and once for 10 min with 0.1 N wash buffer at 42° C. The images were analyzed with Phosphorimager, visualized by Typhoon 9410 Imager (GE Healthcare), and processed with ImageQuant 5.2 software (Molecular Dynamics).

DNA damage and telomere dysfunction assay (TIF). For assessment of global DNA damage cells were fixed and permeabilized with BD CytoFix/Perm reagent following the manufacturer's instructions and incubated with γH2AX antibody (Cell Signaling Technology, Danvers, Mass.) for 1 hr at room temperature followed by 1 hr incubation with Alexa-647 anti-Rabbit secondary antibody (A21244; Life Technologies, Carlsbad, Calif.). Mean fluorescence staining was quantified by flow cytometry. For NAC treatment cells were treated with 1 mM N-Acetyl-N-cysteine (Sigma Aldrich) in PBS for the duration of the experiment. NAC was replaced every 48 h up to day seven.

Measurement of reactive oxygen species. General or mitochondrial specific ROS were measured by flow cytometry H2DCFDA or MitoSoxRed (Invitrogen, Waltham, Mass.) following manufacturer's specifications.

3D tumor spheroid models. 5×10³ cells were seeded in 96-well plates coated with 1% agar in PBS and allowed to grow for 72 h. Spheroids were embedded into rat collagen type I (Corning, Bedford, Mass.) mixture as previously described 60 and treated with drugs for 7 days. Spheroids were stained with Live/Dead cell assay (Invitrogen, Waltham, Mass.) and imaged using a Nikon Inverted TE2000 microscope (Melville, N.Y., USA). Images were processed and merged using Image Pro software (Media Cybernetics, Rockville, Md., USA).

Animal studies. Female and male (5-6 weeks old) NOD/LtSscidIL2Rγ-null mice (NSG) mice were injected subcutaneously with 1×10⁶ NRASmut melanoma cells (WM3000 or M93-047) in a suspension of matrigel (BD Matrigel Basement Membrane Matrix, Growth Factor Reduced)/RPMI media at a ratio of 1:1. Tumor growth was measured twice weekly with digital calipers. Once tumors reached an average volume of 50-100 mm³, mice were randomized into different treatment groups. Randomization was performed for all in vivo studies using Random.org following atmospheric noise algorithm. For single drug studies, 2.5 mg/Kg of 6-thio-dG was administered (i.p, q.d.). For combination studies, same dose of 6-thio-dG was administered alone for the first 7 days (q.d.). Mice were then treated with 12.5 mg/K of Gamitrinib (i.p, q.d.) and 6-thio-dG (ip, q.o.d.) up to 20 days. Tumor volume over time were used to model tumor growth rate in each treatment group. No blinding was done for these studies.

To have at least 80% power with a two-sided type I error rate of 5%, 7 mice per group were used for in vivo drug combination studies. The only criteria for exclusion was health and well-being of the animals. No animals were excluded from the analysis. Tumor growth rates were compared between treatment groups using a linear mixed effect model or mixed-effect spline model with the random effect at individual animal level. A p-value <0.05 was considered statistically significant. For survival studies, treatment was discontinued after 20 days, and animals were followed-up for 10 additional days. For survival analysis, tumor volume endpoint was preset at 1500 mm³ and data represented as Kaplan-Meir curves. All animal studies were approved by the Wistar Institute IACUC. All animal studies were conducted in accordance with NIH animal care and use guidelines, and mice were maintained according to the guidelines of the IACUC of The Wistar Institute.

Statistical Analysis

All experiments were performed at least three independent times unless otherwise indicated; sample size (N) is indicated in the figure legends. Data are expressed as average±SEM unless otherwise indicated. For in vitro experiments with n=3/group, the inventors target 80% power to test a large effect size of 3.1 at a two-sided type I error rate of 5%. Results normally distributed with equal variance between groups were analyzed by unpaired two-tailed Student's t-test. If variance was not similar between groups, student's t-test with unequal variances was applied. Asterisks denote P-value significance: *p<0.05; **p<0.01; ***p<0.005. Sample sizes, statistical tests and p values are indicated in the figures. All statistical analyses were calculated using Stata 14, GraphPad Prism5 software, or Microsoft Excel. For microscope images (spheroids, IF) and immunoblots representative images of three independent experiments are shown.

SUPPLEMENTARY TABLE 1
Genotype of NRAS mutant melanoma cell lines used in the study

| Cell line | NRAS mutation | TERT promoter mutation |
|---|---|---|
| WM3000 | Q61R | C/T (−124, −138, −139) |
| WM3451 | Q61K | T/C, C/T (−246, −139, −138) |
| M93-047 | Q61K | — |
| WM852 | Q61R | C/C, C/T (−246, −139, −138) |
| WM1366 | Q61L | T/C, C/T (−138, −139) |
| WM3629 | G12D | T/C (−124) |
| WM4113 | Q61R | A/C (−57) |

SUPPLEMENTARY TABLE 2
Primers sequences

| Gene | Forward Seq. 5′-3′ | Reverse Seq. 5′-3′ |
|---|---|---|
| GAPDH | TGCCAATGATGACATCAA | GGAGTGGGTGTCGCTGTT |
| | GAA | G |
| TERT | GCCGATTGTGAACATGGA | GCTCGTAGTTGAGCACGC |
| | CTACG | TGAA |
| PGC1 | CTGCTAGCAAGTTTGCCT | AGTGGTGCAGTGACCAAT |
| alpha | CA | CA |
| PGC1 | CAGACAGAACGCCAAGCA | TCGCACTCCTCAATCTCA |
| beta | TC | CC |
| SOD2 | GGCCTACGTGAACAACCT | CCGTTAGGGCTGAGGTTT |
| | GA | GT |
| GPX1 | TATCGAGAATGTGGCGTC | CAAACTGGTTGCACGGGA |
| | CC | AG |
| UCP2 | CCTCTCCCAATGTTGCTC | GGCAAGGGAGGTCATCTG |
| | GT | TC |

Although the disclosure has been described with reference to the above example, it will be understood that modifications and variations are encompassed within the spirit and scope of the disclosure.

### REFERENCES

The following references, to the extent that they provide exemplary procedural or other details supplementary to those set forth herein, are specifically incorporated herein by reference.
1 Artandi S E, DePinho R A. Telomeres and telomerase in cancer. Carcinogenesis 2010; 31: 9-18.
2 Baerlocher G M, Oppliger Leibundgut E, Ottmann OG, Spitzer G, Odenike O, McDevitt MA et al. Telomerase Inhibitor Imetelstat in Patients with Essential Thrombocythemia. N Engl J Med 2015; 373: 920-928.
3 Bagheri S, Nosrati M, Li S, Fong S, Torabian S, Rangel J et al. Genes and pathways downstream of telomerase in melanoma metastasis. Proc Natl Acad Sci U S A 2006; 103: 11306-11311.
4 Bryan C, Rice C, Hoffman H, Harkisheimer M, Sweeney M, Skordalakes E. Structural Basis of Telomerase Inhibition by the Highly Specific BIBR1532. Structure 2015; 23: 1934-1942.
5 Ceccarelli M, Barthel F P, Malta T M, Sabedot T S, Salama S R, Murray B A et al. Molecular Profiling Reveals Biologically Discrete Subsets and Pathways of Progression in Diffuse Glioma. Cell 2016; 164: 550-563.
6 Chae Y C, Caino M C, Lisanti S, Ghosh J C, Dohi T, Danial N N et al. Control of tumor bioenergetics and survival stress signaling by mitochondrial HSP90s. Cancer Cell 2012; 22: 331-344.
7 Chiba K, Lorbeer F K, Shain A H, McSwiggen D T, Schruf E, Oh A et al. Mutations in the promoter of the telomerase gene TERT contribute to tumorigenesis by a two-step mechanism. Science 2017; 357: 1416-1420.
8 Choi J, Southworth L K, Sarin K Y, Venteicher A S, Ma W, Chang W et al. TERT promotes epithelial proliferation through transcriptional control of a Myc- and Wnt-related developmental program. PLoS Genet 2008; 4: e10.
9 Damm K, Hemmann U, Garin-Chesa P, Hauel N, Kauffmann I, Priepke H et al. A highly selective telomerase inhibitor limiting human cancer cell proliferation. EMBO J 2001; 20: 6958-6968.
10 Easty D J, Gray S G, O'Byrne K J, O′Donnell D, Bennett D C. Receptor tyrosine kinases and their activation in melanoma. Pigment Cell Melanoma Res 2011.
11 Fleisig H B, Hukezalie K R, Thompson C A, Au-Yeung T T, Ludlow A T, Zhao C R et al. Telomerase reverse transcriptase expression protects transformed human cells against DNA-damaging agents, and increases tolerance to chromosomal instability. Oncogene 2016; 35: 218-227.
12 Garcia-Beccaria M, Martinez P, Mendez-Pertuz M, Martinez S, Blanco-Aparicio C, Canamero M et al. Therapeutic inhibition of TRF1 impairs the growth of p53-deficient K-RasG12V-induced lung cancer by induction of telomeric DNA damage. EMBO Mol Med 2015; 7: 930-949.
13 Greider C W. Telomerase activity, cell proliferation, and cancer. Proceedings of the National Academy of Sciences of the United States of America 1998; 95: 90-92.
14 Griewank K G, Murali R, Puig-Butille J A, Schilling B, Livingstone E, Potrony M et al. TERT promoter mutation status as an independent prognostic factor in cutaneous melanoma. J Natl Cancer Inst 2014; 106.
15 Gupta R, Dong Y, Solomon P D, Wettersten H I, Cheng C J, Min J N et al. Synergistic tumor suppression by combined inhibition of telomerase and CDKN1A. Proc Natl Acad Sci U S A 2014; 111: E3062-3071.
16 Haq R, Shoag J, Andreu-Perez P, Yokoyama S, Edelman H, Rowe G C et al. Oncogenic BRAF regulates oxidative metabolism via PGC1 alpha and MITF. Cancer Cell 2013; 23: 302-315.
17 Hayward N K, Wilmott J S, Waddell N, Johansson P A, Field M A, Nones K et al. Whole-genome landscapes of major melanoma subtypes. Nature 2017.
18 Hodis E, Watson I R, Kryukov G V, Arold S T, Imielinski M, Theurillat J P et al. A landscape of driver mutations in melanoma. Cell 2012; 150: 251-263.
19 Horn S, Figl A, Rachakonda P S, Fischer C, Sucker A, Gast A et al. TERT promoter mutations in familial and sporadic melanoma. Science 2013; 339: 959-961.
20 Hrdlickova R, Nehyba J, Bose H R, Jr. Alternatively spliced telomerase reverse transcriptase variants lacking telomerase activity stimulate cell proliferation. Mol Cell Biol 2012; 32: 4283-4296.
21 Hu J, Hwang S S, Liesa M, Gan B, Sahin E, Jaskelioff M et al. Antitelomerase therapy provokes ALT and mitochondrial adaptive mechanisms in cancer. Cell 2012; 148: 651-663.
22 Huang F W, Hodis E, Xu M J, Kryukov G V, Chin L, Garraway L A. Highly recurrent TERT promoter mutations in human melanoma. Science 2013; 339: 957-959.
23 Indran I R, Hande M P, Pervaiz S. hTERT overexpression alleviates intracellular ROS production, improves mitochondrial function, and inhibits ROS-mediated apoptosis in cancer cells. Cancer Res 2011; 71: 266-276.
24 Jafri M A, Ansari S A, Alqahtani M H, Shay J W. Roles of telomeres and telomerase in cancer, and advances in telomerase-targeted therapies. Genome Med 2016; 8: 69.
25 Jakob J A, Bassett R L, Jr., Ng C S, Curry J L, Joseph R W, Alvarado G C et al. NRAS mutation status is an independent prognostic factor in metastatic melanoma. Cancer 2012; 118: 4014-4023.
26 Johnson D B, Smalley K S, Sosman J A. Molecular pathways: targeting NRAS in melanoma and acute myelogenous leukemia. Clin Cancer Res 2014; 20: 4186-4192.
27 Johnson D B, Puzanov I. Treatment of NRAS-mutant melanoma. Curr Treat Options Oncol 2015; 16: 15.

28 Joseph I, Tressler R, Bassett E, Harley C, Buseman C M, Pattamatta P et al. The telomerase inhibitor imetelstat depletes cancer stem cells in breast and pancreatic cancer cell lines. Cancer Res 2010; 70: 9494-9504.
29 Kang B H, Siegelin M D, Plescia J, Raskett C M, Garlick D S, Dohi T et al. Preclinical characterization of mitochondria-targeted small molecule hsp90 inhibitors, gamitrinibs, in advanced prostate cancer. Clin Cancer Res 2010; 16: 4779-4788.
30 Koh C M, Khattar E, Leow S C, Liu C Y, Muller J, Ang W X et al. Telomerase regulates MYC-driven oncogenesis independent of its reverse transcriptase activity. J Clin Invest 2015; 125: 2109-2122.
31 Kops G J, Dansen T B, Polderman P E, Saarloos I, Wirtz K W, Coffer P J et al. Forkhead transcription factor FOXO3a protects quiescent cells from oxidative stress. Nature 2002; 419: 316-321.
32 Kovalenko O A, Caron M J, Ulema P, Medrano C, Thomas A P, Kimura M et al. A mutant telomerase defective in nuclear-cytoplasmic shuttling fails to immortalize cells and is associated with mitochondrial dysfunction. Aging Cell 2010; 9: 203-219.
33 Li Y, Tergaonkar V. Noncanonical functions of telomerase: implications in telomerase-targeted cancer therapies. Cancer Res 2014; 74: 1639-1644.
34 Li Y, Cheng H S, Chng W J, Tergaonkar V. Activation of mutant TERT promoter by RAS-ERK signaling is a key step in malignant progression of BRAF-mutant human melanomas. Proc Natl Acad Sci U S A 2016; 113: 14402-14407.
35 Listerman I, Sun J, Gazzaniga F S, Lukas J L, Blackburn E H. The major reverse transcriptase-incompetent splice variant of the human telomerase protein inhibits telomerase activity but protects from apoptosis. Cancer Res 2013; 73: 2817-2828.
36 Maertens O, Johnson B, Hollstein P, Frederick D T, Cooper Z A, Messiaen L et al. Elucidating distinct roles for NF1 in melanomagenesis. Cancer Discov 2013; 3: 338-349.
37 Martinez P, Blasco M A. Telomeric and extra-telomeric roles for telomerase and the telomere-binding proteins. Nature reviews Cancer 2011; 11: 161-176.
38 Masutomi K, Possemato R, Wong J M, Currier J L, Tothova Z, Manola J B et al. The telomerase reverse transcriptase regulates chromatin state and DNA damage responses. Proc Natl Acad Sci U S A 2005; 102: 8222-8227.
39 Mender I, Gryaznov S, Dikmen Z G, Wright W E, Shay J W. Induction of telomere dysfunction mediated by the telomerase substrate precursor 6-thio-2′-deoxyguanosine. Cancer Discov 2015; 5: 82-95.
40 Mender I, Shay J W. Telomerase Repeated Amplification Protocol (TRAP). Bio Protoc 2015; 5.
41 Nazarian R, Shi H, Wang Q, Kong X, Koya R C, Lee H et al. Melanomas acquire resistance to B-RAF(V600E) inhibition by RTK or N-RAS upregulation. Nature 2010; 468: 973-977.
42 Nissan M H, Pratilas C A, Jones A M, Ramirez R, Won H, Liu C et al. Loss of NF1 in cutaneous melanoma is associated with RAS activation and MEK dependence. Cancer Res 2014; 74: 2340-2350.
43 Nosrati M, Li S, Bagheri S, Ginzinger D, Blackburn E H, Debs R J et al. Antitumor activity of systemically delivered ribozymes targeting murine telomerase RNA. Clin Cancer Res 2004; 10: 4983-4990.
44 Olmos Y, Valle I, Borniquel S, Tierrez A, Soria E, Lamas S et al. Mutual dependence of Foxo3a and PGC-lalpha in the induction of oxidative stress genes. J Biol Chem 2009; 284: 14476-14484.
45 Postow M A, Chapman P B. A step forward for patients with NRAS-mutant melanoma. Lancet Oncol 2017; 18: 414-415.
46 Puri N, Eller M S, Byers H R, Dykstra S, Kubera J, Gilchrest B A. Telomere-based DNA damage responses: a new approach to melanoma. FASEB J 2004; 18: 1373-1381.
47 Queisser A, Heeg S, Thaler M, von Werder A, Opitz O G. Inhibition of telomerase induces alternative lengthening of telomeres during human esophageal carcinogenesis. Cancer Genet 2013; 206: 374-386.
48 Roesch A, Vultur A, Bogeski I, Wang H, Zimmermann K M, Speicher D et al. Overcoming Intrinsic Multidrug Resistance in Melanoma by Blocking the Mitochondrial Respiratory Chain of Slow-Cycling JARID1B(high) Cells. Cancer Cell 2013; 23: 811-825.
49 Santos J H, Meyer J N, Van Houten B. Mitochondrial localization of telomerase as a determinant for hydrogen peroxide-induced mitochondrial DNA damage and apoptosis. Human molecular genetics 2006; 15: 1757-1768.
50 Sarin K Y, Cheung P, Gilison D, Lee E, Tennen R I, Wang E et al. Conditional telomerase induction causes proliferation of hair follicle stem cells. Nature 2005; 436: 1048-1052.
51 Sharma N K, Reyes A, Green P, Caron M J, Bonini M G, Gordon D M et al. Human telomerase acts as a hTR-independent reverse transcriptase in mitochondria. Nucleic Acids Res 2012; 40: 712-725.
52 Shay J W. Role of Telomeres and Telomerase in Aging and Cancer. Cancer Discov 2016.
53 Shin K H, Kang M K, Dicterow E, Kameta A, Baluda M A, Park N H. Introduction of human telomerase reverse transcriptase to normal human fibroblasts enhances DNA repair capacity. Clin Cancer Res 2004; 10: 2551-2560.
54 Siegelin M D, Dohi T, Raskett C M, Orlowski G M, Powers C M, Gilbert C A et al. Exploiting the mitochondrial unfolded protein response for cancer therapy in mice and human cells. J Clin Invest 2011; 121: 1349-1360.
55 Smith L L, Coller H A, Roberts J M. Telomerase modulates expression of growth-controlling genes and enhances cell proliferation. Nat Cell Biol 2003; 5: 474-479.
56 Tefferi A, Lasho T L, Begna K H, Patnaik M M, Zblewski D L, Finke C M et al. A Pilot Study of the Telomerase Inhibitor Imetelstat for Myelofibrosis. N Engl J Med 2015; 373: 908-919.
57 Vallarelli A F, Rachakonda P S, Andre J, Heidenreich B, Riffaud L, Bensussan A et al. TERT promoter mutations in melanoma render TERT expression dependent on MAPK pathway activation. Oncotarget 2016; 7: 53127-53136.
58 Van Allen E M, Wagle N, Sucker A, Treacy D J, Johannessen C M, Goetz E M et al. The genetic landscape of clinical resistance to RAF inhibition in metastatic melanoma. Cancer Discov 2014; 4: 94-109.
59 Viceconte N, Dheur M S, Majerova E, Pierreux C E, Baurain J F, van Baren N et al. Highly Aggressive Metastatic Melanoma Cells Unable to Maintain Telomere Length. Cell reports 2017; 19: 2529-2543.
60 Villanueva J, Vultur A, Lee J T, Somasundaram R, Fukunaga-Kalabis M, Cipolla A K et al. Acquired resistance to BRAF inhibitors mediated by a RAF kinase switch in melanoma can be overcome by cotargeting MEK and IGF-1R/PI3K. Cancer Cell 2010; 18: 683-695.
61 Wong D J, Ribas A. Targeted Therapy for Melanoma. Cancer Treat Res 2016; 167: 251-262.
62 Zhang G, Frederick D T, Wu L, Wei Z, Krepler C, Srinivasan S et al. Targeting mitochondrial biogenesis to overcome drug resistance to MAPK inhibitors. J Clin Invest 2016; 126: 1834-1856.

1. A method of treating a cancer cell or a tumor having an NRAS mutation comprising contacting the tumor with 6-thio-2′-deoxy-guanosine (6-thio-dG).

2. The method of claim 1, wherein the method further comprises contacting the cancer cells or tumor with a mitochondrial inhibitor prior to treatment with 6-thio-dG.

3. The method of claim 2, wherein the mitochondrial inhibitor is Gamitrinib.

4. The method of claim 1, wherein the cancer or tumor is melanoma.

5. The method of claim 4, wherein the melanoma is metastatic melanoma.

6. The method of claim 1, wherein the 6-thio-dG is administered by an oral, intratumoral, intravenous or intrathecal route.

7. The method of claim 1, wherein the cancer or tumor is selected from breast, prostate, colon, liver, kidney, melanoma, skin cancer, head and neck, brain, lung, bone, hematopoietic cancer, leukemias, and pancreatic cancer.

8. The method of claim 7, wherein the cancer is metastatic.

9. A pharmaceutical composition comprising 6-thio-dG and a mitochondrial inhibitor.

10. The pharmaceutical composition of claim 9, wherein the mitochondrial inhibitor is Gamitrinib.

11. The method of claim 1, wherein the method further comprises contacting the cancer cells or tumor with a mitochondrial inhibitor simultaneous with treatment with 6-thio-dG.

12. The method of claim 1, wherein the method further comprises contacting the cancer cells or tumor with a mitochondrial inhibitor after treatment with 6-thio-dG.

13. The method of claim 4, wherein the NRAS mutation is Q61R, Q61K, G61L, or G12D.

14. The method of claim 5, wherein the NRAS mutation is Q61R, Q61K, G61L, or G12D.

15. The method of claim 2, wherein the mitochondrial inhibitor is phenformin.

16. The pharmaceutical composition of claim 9, wherein the mitochondrial inhibitor is phenformin.
